(19) United States
(12) Patent Application Publication
(10) Pub. No.: US 2025/0128053 A1
(43) Pub. Date: Apr. 24, 2025

(54) SYSTEM AND DEVICE FOR PURGING BLOOD PUMPS

(71) Applicant: ABIOMED, Inc., Danvers, MA (US)

(72) Inventors: Ahmad El Katerji, Danvers, MA (US); Daniel Podlisny, Danvers, MA (US)

(73) Assignee: ABIOMED, Inc., Danvers, MA (US)

(21) Appl. No.: 18/924,293

(22) Filed: Oct. 23, 2024

Related U.S. Application Data
(60) Provisional application No. 63/545,336, filed on Oct. 23, 2023.

(51) Int. Cl. A61M 60/90; A61M 60/122

(52) U.S. Cl. CPC A61M 60/90; A61M 60/122; A61M 2205/04; A61M 2205/103; A61M 2205/18; A61M 2205/3327; A61M 2205/3331; A61M 2205/3379; A61M 2205/50; A61M 2209/00

(57) ABSTRACT

Various devices, systems, and techniques are provided for purging blood pumps. A purge apparatus may be provided that may include a housing having a hinged lid defining a first surface of the housing. The hinged lid may be configured to allow access to an internal volume of space of the housing. The housing may have an opening on a second surface opposite the first surface. The housing may define a tubing channel configured to receive at least a portion of a tube. The purge apparatus may include a pumping mechanism configured to be operably connected to a motor through the opening. The pumping mechanism may be disposed within the internal volume of space of the housing. The pumping mechanism may be configured to, during operation, compress the tube extending through the purge apparatus so as to continuously maintain a positive pressure downstream of the pumping mechanism.

### CROSS-REFERENCE TO RELATED APPLICATIONS

The present application claims priority to U.S. provisional patent applications 63/545,336filed Oct. 23, 2023, which is incorporated by reference herein by its entirety.

### TECHNICAL FIELD

The present disclosure is drawn to systems, methods and techniques for purging blood pumps.

### BACKGROUND

Blood pumps of different types are known, such as axial blood pumps, centrifugal blood pumps, or mixed-type blood pumps, where the blood flow is caused by both axial and radial forces. One example of a blood pump is the IMPELLA® line of blood pumps (e.g., IMPELLA® 2.5, IMPELLA® CP, IMPELLA® 5.5, etc.) which are products of Abiomed of Danvers, MA. Intravascular blood pumps are inserted into a patient's vessel such as the aorta by means of a catheter.

In some pump designs, a purge fluid may be deployed to keep blood from entering the mechanism and to mitigate the effects of blood on the pump mechanisms, an anticoagulant such as heparin (typically the sodium salt of heparin). The heparin is thought to keep the blood from coagulating in the gap between pump components such as an impeller shaft and the housing. Heparin is a commonly used anticoagulant typically administered in controlled dosages. The purge fluid may be delivered by a purge cassette (that is coupled to a purge fluid source) that controls the flow of the purge fluid to a blood pump catheter through a filter assembly and internal purge lumen that carries the purge fluid through the catheter to a purge channel in the motor assembly. The flow of the purge fluid is regulated by an automated controller.

However, changing such a purge cassette may be problematic for a treatment, as doing so requires various time-consuming steps, such as purging the cassette of air, before the purge fluid can again be sent to the blood pump.

### BRIEF SUMMARY

In various aspects, a purge apparatus may be provided. The purge apparatus may include a housing. The housing may have a hinged lid. The hinged lid may define a first surface of the housing. The hinged lid may be configured to allow access to an internal volume of space of the housing. The housing may have an opening on a second surface opposite the first surface. The housing may define a tubing channel. The tubing channel may be configured to receive at least a portion of a tube. The purge apparatus may include a pumping mechanism. The pumping mechanism may be configured to be operably coupled to a motor through the opening. The pumping mechanism may be disposed within the internal volume of space of the housing. The pumping mechanism may be configured to, during operation, compress the tube extending through the purge apparatus so as to continuously maintain a positive pressure downstream of the pumping mechanism.

In some embodiments, the tubing channel may be configured to, during operation, allow the tube to be pressed against a surface of the hinged lid.

In some embodiments, the pumping mechanism may include a plurality of rollers or wheels. The plurality of rollers or wheels may be operably coupled together. Each roller or wheel may be spaced an equal distance apart. The plurality of rollers or wheels may be configured to move within the housing such that, during operation, at least one roller or wheel compresses the tube against an internal surface of the housing at any point in time.

In some embodiments, the plurality of rollers or wheels may be coupled together via a belt or chain. The belt or chain may be operably coupled to a connector gear or wheel. The connector gear or wheel may be operably coupled to the motor through the opening. The equal distance each roller or wheel may be separated by may be a distance longer than a circumference of the connector gear or wheel. The equal distance each roller or wheel may be separated by may be a distance less than or equal to a circumference of the connector gear or wheel. The connector gear or wheel may be configured to receive a drive shaft passing through the opening.

In some embodiments, each roller or wheel may be an identical shape and size.

In some embodiments, the pumping mechanism may include a pump wheel. The pump wheel may have a plurality of extruding portions. Each extruding portion may have an equal pitch. The pump wheel may be configured to rotate within the housing such that, during operation, at least one extruding portion compresses the tube against an internal surface of the housing at any point in time.

In some embodiments, a circumferential length of each extruding portion may be longer than a distance separating each extruding portion from an adjacent extruding portion. In some embodiments, a circumferential length of each extruding portion may be less than or equal to a distance separating each extruding portion from an adjacent extruding portion. In some embodiments, a radial height of each extruding portion may be greater than an outer diameter of the tube. In some embodiments, a radial height of each extruding portion may be less than or equal to an outer diameter of the tube. In some embodiments, the tubing channel may be configured to define a path extending at least 180 degrees around a circumference of the pump wheel. In some embodiments, the pump wheel may be configured to be operably coupled to the motor via one or more gears disposed within the housing. In some embodiments, a first gear of the one or more gears may be configured to receive a drive shaft extending through the opening. The first gear may not be vertically centered within the housing.

In some embodiments, the pumping mechanism may include a plurality of rigid compression members disposed along a shaft. The plurality of rigid compression members may be configured to provide a pattern of compression to the tube as the shaft rotates. The shaft may be configured to be coupled to one or more worm threads on a drive shaft to cause the shaft to rotate. In some embodiments, the pattern of compression may be a sinusoidal pattern of compression. In some embodiments, each rigid compression member may be in contact with at least one other rigid compression member. In some embodiments, a major axis of each rigid compression member may be rotated a predetermined angle relative to a major axis of an adjacent rigid compression member. The predetermined angle may be less than 90 degrees. The shaft may extend through a geometric center of each rigid compression member. The shaft may extend through a centroid of each rigid compression member. The shaft may extend through a point offset from the geometric center of each rigid compression member. The shaft may be cylindrical. The shaft may be segmented.

In various aspects, a system may be provided. The system may include one or more processors. The system may include an electric motor operably coupled to the one or more processors. The system may include a housing disposed around the one or more processors and the electric motor. A drive shaft of the electric motor may extend through an outer surface of the housing (e.g., through an opening in the housing). The system may include a purge apparatus as disclosed herein. The purge apparatus may be coupled to the housing such that the drive shaft extends through the opening of the purge apparatus. The pumping mechanism of the purge apparatus may be operably coupled to the drive shaft.

In some embodiments, the system may include a purge bag. The purge bag may contain a purge fluid. The purge bag may be operably coupled to a tube. A portion of the tube may be disposed in a tubing channel of the purge apparatus. The system may include a blood pump operably coupled to the purge bag via the tube.

In some embodiments, the one or more processors may be configured to, collectively, determine a pressure of a fluid in the tube based on a current of the electric motor. In some embodiments, determining the pressure of the fluid in the tube may be based on a change in the current of the electric motor. In some embodiments, determining the pressure of the fluid in the tube may be based on the current of the electric motor and information from at least one other sensor. The one or more processors may be configured to, collectively, generate an alert or alarm based on the pressure of the fluid in the tube. The one or more processors may be configured to, collectively, generate an alert or alarm if the pressure of the fluid in the tube is outside a predetermined range. The one or more processors may be configured to, collectively, generate an alert or alarm when the current of the electric motor changes from a first value to a second value, the second value being lower than the first value. The one or more processors may be configured to, collectively, determine if a liquid is present in the tube based on a current of the electric motor. The one or more processors may be configured to, collectively, determine a current of the electric motor once every T seconds, where T≥1. In some embodiments, determining the current may include determining an average current of the electric motor over T seconds. In some embodiments, determining the current may include determining a maximum current of the electric motor over T seconds. In some embodiments, determining the current may include determining a minimum current of the electric motor over T seconds.

In some embodiments, the one or more processors may be configured to, collectively, adjust a current of the electric motor based on a determined current of the electric motor. In some embodiments, adjusting a current of the electric motor may include increasing the current if the determined current is below a lower threshold. Adjusting the current may include decreasing the current if the determined current is above an upper threshold.

In some embodiments, the one or more processors may be configured to, collectively, determine an amount of a liquid fluid passing through the pumping mechanism based on a number of rotations of a wheel of the pumping mechanism. The one or more processors may be configured to, collectively, monitor a total amount of liquid fluid passing through the pumping mechanism over a period of time based on the number of rotations of the wheel of the pumping mechanism The one or more processors may be configured to, collectively, generate an alert or alarm when the total amount of liquid fluid meets or exceeds a predetermined threshold. The predetermined threshold may be based on a volume of a source of the liquid fluid. The one or more processors may be configured to, collectively, display the total amount of fluid on a display operably coupled to the one or more processors. The one or more processors may be configured to, collectively, stop the pumping mechanism when the current of the electric motor changes from a first value to a second value, the second value being lower than the first value.

In various aspects, a method may be provided. The method may include rotating a drive shaft coupled to an electric motor, causing a pumping mechanism to move liquid fluid from a fluid source through a tube. The pumping mechanism may be disposed in a housing. At least a portion of the tube may be disposed within the housing. The drive shaft may extend through a channel in the housing.

The method may include measuring a current to the electric motor while the drive shaft is rotating. The method may include determining a pressure in the tube based on the current to the electric motor. The method may include generating an alert or alarm if the determined pressure in the tube is outside a predetermined range. The method may include adjusting a current to the electric motor if the measured current is outside a predetermined range.

In various aspects, a method may be provided. The method may include rotating a drive shaft coupled to an electric motor, causing a pumping mechanism to move liquid fluid from a fluid source through a tube. The pumping mechanism may be disposed in a housing. At least a portion of the tube may be disposed within the housing. The drive shaft may extend through a channel in the housing.

The method may include determining a number of rotations of a wheel of the pumping mechanism during a period of time. The method may include determining a volume of liquid fluid passing through the tube based on the number of rotations. The method may include generating an alert or alarm if the volume of liquid fluid meets or exceeds a predetermined threshold.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a blood pump within a patient, coupled to a purge fluid source via a controller.

FIG. 2A is an illustration of a controller including a purge apparatus.

FIG. 2B is an illustration of a cross-sectional top view of an embodiment of a system.

FIG. 3A is an illustration of a cross-sectional front view of an embodiment of a purge apparatus.

FIG. 3B is an illustration of a cross-sectional top view of another embodiment of a purge apparatus.

FIG. 3C-3D are illustrations of cross-sectional front views of other embodiments of a purge apparatus.

FIG. 3E-3F are illustrations of axial views of an embodiments of drive shafts for embodiments of a purge apparatus such as those shown in FIG. 3D.

FIG. 3G is an illustration of a segmented drive shaft.

FIG. 4 is a flowchart of a method.

FIG. 5 is a flowchart of a method.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

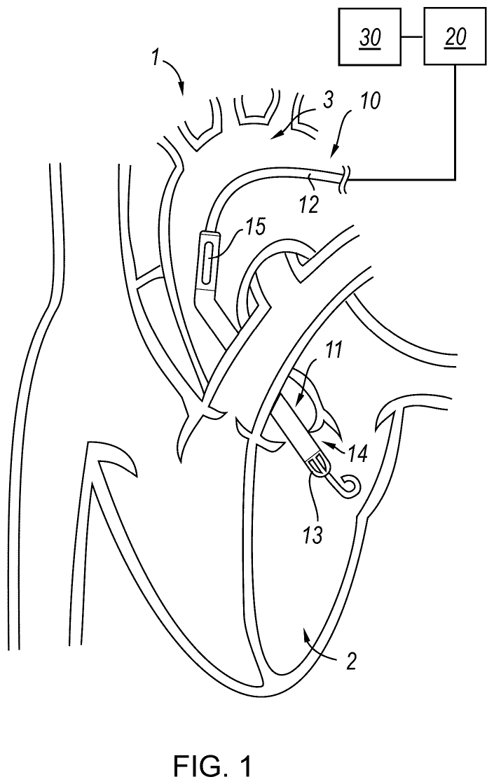

Referring to FIG. 1, in various aspects, a blood pump 10 may be positioned in a patient, such as within a heart 1. In some embodiments, the blood pump may include a pump portion 11 coupled to a distal end of a catheter 12. The pump portion may include a blood inlet 13 configured to allow blood to flow into the blood pump, through a cannula 14 which may include, e.g., an impeller (not shown), and out through a blood outlet 15. The blood inlet and blood outlet may be located in different areas of the patient; for example, the blood inlet may be positioned in the left ventricle 2, and the blood outlet may be positioned in an aortic arch 3.

The blood pump may be controlled via a controller 20. The controller may be coupled to a purge fluid source 30. The controller may provide purge fluid from the purge fluid source to the pump portion 11 via, e.g., a lumen in catheter 12. The controller 20 may include a purge apparatus 100. The purge apparatus may be configured to receive a purge cassette.

Conventionally, the controller may include a removable replacement purge cassette that provides all the necessary components for controlling fluid flow to the blood pump. Changing conventional purge cassettes may be problematic for treatment, as doing so requires various time-consuming steps, such as purging the cassette of air, before the purge fluid can again be sent to the blood pump. The present disclosure provides techniques for simplifying the process for providing a purge fluid to a blood pump. Such approaches also allow for simplification of the purge cassette.

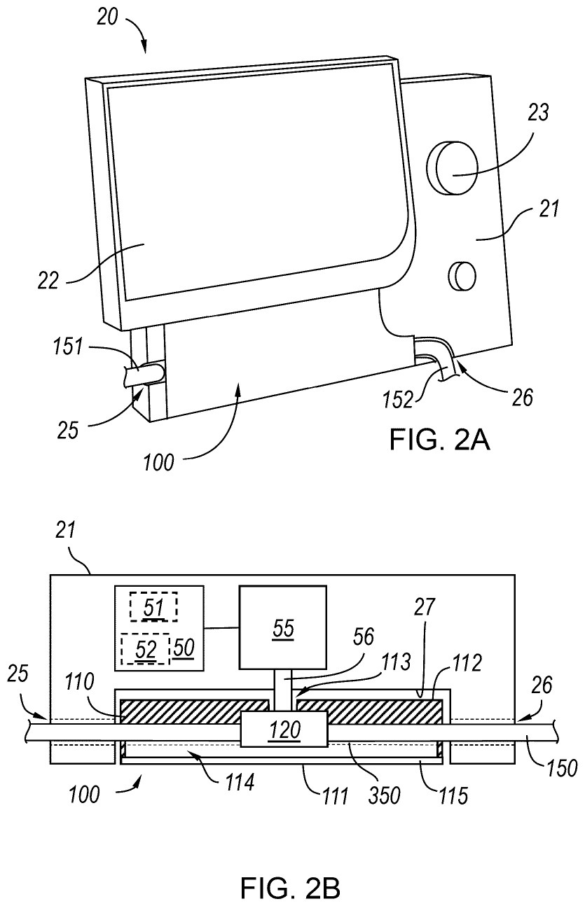

Referring to FIG. 2A, a controller 20 may include a housing 21, a display 22, and one or more buttons or controls 23. A purge apparatus 100 may be removably coupled to the controller. An upstream portion 151 of a tube 150 may exit the purge apparatus at a first location 25, such as a first port or channel adapted to allow the tube to exit the housing. A downstream portion 152 of the tube 150 may exit the purge apparatus at a second location 26, such as a second port or channel adapted to allow the tube to exit the housing.

In various aspects, a purge apparatus may be provided. Referring to FIG. 2B, a purge apparatus 100 may include a housing 110. The housing may have a hinged lid 115 defining a first surface 111 of the housing 110. The hinged lid 115 may be configured to allow access to an internal volume of space 114 of the housing. The housing 110 may have an opening 113 on a second surface 112 of the housing 110 opposite the first surface 111. The housing 110 may define a tubing channel 350 configured to receive at least a portion of a tube.

The purge apparatus may include a pumping mechanism 120 configured to be operably coupled to a motor 55 through the opening. The pumping mechanism 120 may be coupled to a drive shaft 56 of the motor 55. The controller 20 may be configured to control the speed of the motor 55. In this manner, the controller 20 may increase or decrease the speed at which the purge fluid is delivered to a blood pump.

The controller 20 may include circuitry 50, which may include one or more processor(s), for controlling the various components of the system (such as the motors, displays, blood pumps, etc.). The circuitry may monitor the current to the motor for drops in current which may indicate air in the line. The controller may further include warning sounds, lights, or indicators to alert an operator of disconnects, kinks, or breaks in the purge tubing, which may result in the introduction of air into the line.

The controller 20 may include at least one sensor 52. The at least one sensor 52 may be a current sensor. The current sensor may be used to measure current consumption by the motor.

The pumping mechanism 120 may be disposed within the internal volume of space 114 of the housing. The pumping mechanism may be configured to, during operation, compress the tube 150 extending through the purge apparatus so as to continuously maintain a positive pressure downstream of the pumping mechanism.

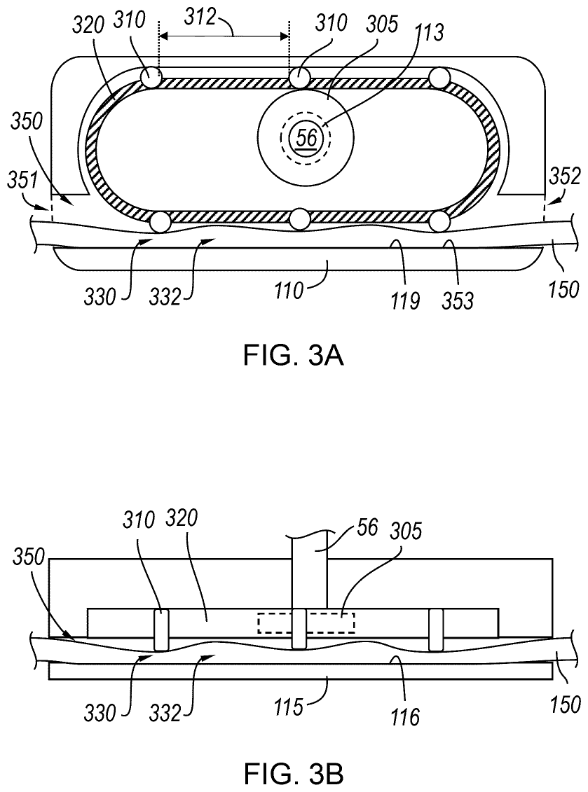

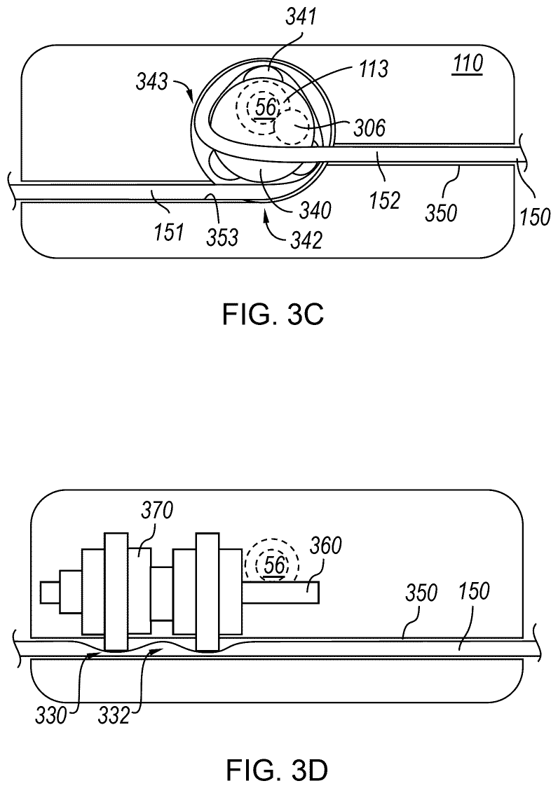

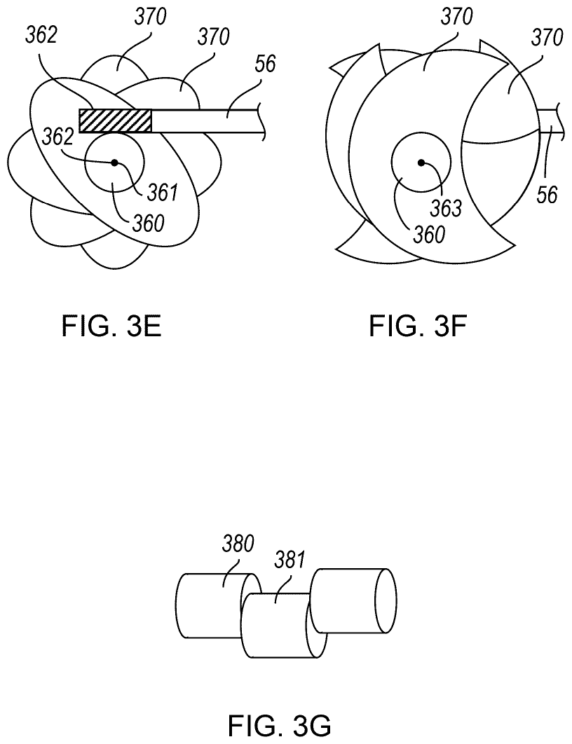

As seen in the front view presented in FIG. 3A, the tubing channel may be configured to, during operation, allow at least a portion of the tube to be pressed against a portion of the tubing channel 350 (shown here as being pressed against an internal surface 119 of the housing), within the tubing channel 350. The pressing of the at least a portion of the tube may be caused by a roller, wheel, extruding portions or rigid compression members (as seen in FIGS. 3A-3D).

FIG. 3B provides a modified version of this embodiment, where rather than pressing the tube against (for example) a bottom surface, the tubing channel is configured to dispose the tube such that the rollers or wheels 310 compress parts of the tube against the hinged lid 315, such as against an internal surface 116 of the hinged lid. Thus, the tubing channel may be configured to, during operation, allow at least a portion of the tube to be pressed against at least a portion of a surface of the hinged lid 115.

Referring back to FIG. 3A, the pumping mechanism may include a plurality of rollers or wheels 310. The plurality of rollers or wheels may be operably coupled together. In some embodiments, each roller or wheel may be spaced a distance 312 apart from an adjacent roller or wheel. This distance 312 may be an equal distance. In some embodiments, at least one roller or wheel is separated from an adjacent roller or wheel by a distance 312 that is different from a distance separating a different pair of adjacent rollers of wheels.

The distance 312 each roller or wheel may be separated by may be a distance longer than a circumference of the connector gear or wheel 305. The distance each roller or wheel may be separated by may be a distance less than or equal to a circumference of the connector gear or wheel. The connector gear or wheel may be configured to receive a drive shaft passing through the opening. The distance between each roller or wheel may be selected to achieve a predetermined flow rate of the purge fluid or to achieve a predetermined pressure of the purge fluid.

In some embodiments, each roller or wheel may be an identical shape and size. In some embodiments, at least one roller or wheel may be a different shape and/or size from a different roller or wheel. In some embodiments, the cross-section of each roller or wheel is circular. In some embodiments, the cross-section of each roller or wheel may be non-circular (such as, e.g., oblong or polygonal, such as hexagonal or octagonal). The shape and size of each roller or wheel may be selected to achieve a predetermined flow rate of the purge fluid or to achieve a predetermined pressure of the purge fluid.

The plurality of rollers or wheels may be configured to move within the housing such that, during operation, at least one roller or wheel compresses the tube against an internal surface 119 of the housing 110 (such as against a surface 353 defining the tubing channel) at any point in time. The plurality of rollers or wheels may be coupled together via a flexible belt or chain 320. The belt or chain may be operably coupled to a connector gear or wheel 305. The connector gear or wheel 305 may be operably coupled to the motor through the opening. In some embodiments, the connector gear or wheel 305 may be directly connected to the drive shaft. In some embodiments, the connector gear or wheel may be indirectly coupled to the drive shaft, e.g., via one or more additional gears or wheels.

The tube 150 may be disposed within a tubing channel 350, having a tubing channel inlet 351 and a tubing channel outlet 352. The apparatus may be configured such that there is at least one compressed portion of the tube 330 and at least one uncompressed portion of the tube 332 within the tubing channel 350. The apparatus may be configured such that there is an uncompressed portion of the tube 332 between two compressed portions of the tube 330 within the tubing channel.

In some aspects, the pumping mechanism may comprise a pump wheel that interacts with the tube. Referring to FIG. 3C, a pump wheel 340 may have a plurality of extruding portions 341. The pump wheel may be configured to rotate within the housing such that, during operation, at least one extruding portion is compressing the tube at any point in time. For example, the plurality of extruding portions may be configured to compress a portion of the tube 150 against, e.g., an internal surface of the housing, such as against surface 353 defining the tubing channel 350.

In some embodiments, each extruding portion may have an equal pitch. In some embodiments, at least one pair of extruding portions may have a different pitch as compared to a different pair of extruding portions. In some embodiments, a circumferential length of each extruding portion may be longer than a distance separating each extruding portion from an adjacent extruding portion. In some embodiments, a circumferential length of each extruding portion may be less than or equal to a distance separating each extruding portion from an adjacent extruding portion. In some embodiments, a radial height of each extruding portion may be greater than an outer diameter of the tube. In some embodiments, a radial height of each extruding portion may be less than or equal to an outer diameter of the tube.

The tubing channel may be configured to define a path extending at least 180 degrees around a circumference of the pump wheel. As seen in FIG. 3C, the upstream portion 152 of the tube 152 extends towards the point 152 at which it contacts the pump wheel 340. The tubing channel then wraps around the pump wheel, for almost 270 degrees, until it reaches a point where the tubing exits the tubing channel and passes around the pump wheel, whereupon the downstream portion 152 of the tube 150 may then re-enter the tubing channel 350.

In some embodiments, the pump wheel 340 may be directly connected to the drive shaft. In some embodiments, the pump wheel may be indirectly connected to the drive shaft, e.g., via one or more additional gears or wheels 306 disposed within the housing. A first gear of the additional gears may be configured to receive a drive shaft extending through the opening. A first gear of the additional gears may be configured to be coupled to the drive shaft. The first gear may not be vertically centered within the housing.

Referring to FIGS. 3D-3E, a pumping mechanism may include a plurality of rigid compression members 370 disposed along a shaft 360. The plurality of rigid compression members may be configured to provide a pattern of compression of tube 150 as the shaft rotates (see, e.g., compressed 330 and uncompressed 332 sections in FIG. 3D).

The shaft 360 may be configured to be coupled to one or more worm threads 362 on a drive shaft 56 to cause the shaft to rotate. In some embodiments, the pattern of compression may be a sinusoidal pattern of compression. In some embodiments, the pattern of compression may be a square wave pattern of compression.

In some embodiments, each rigid compression member 370 may be in contact with at least one other rigid compression member. In some embodiments, each rigid compression member 370 may be separated from every adjacent rigid compression member (e.g., in a distance parallel to the axis 361 of the shaft 360. In some embodiments, each rigid compression member may be in contact with at least one other rigid compression member, and at least one rigid compression member is separated from an adjacent rigid compression member (for example, in FIG. 3D, there could be a space between the fourth and fifth rigid compression members).

As shown in FIG. 3D, in some embodiments, all rigid compression members are on one side of the drive shaft 56 in a direction parallel to the direction of the tube. In FIG. 3D, the rigid compression members 370 are shown as being to the left when viewing the apparatus from the front (or in an “upstream” direction). The rigid compression members 370 could very easily be converted to being to the right (or in the “downstream” direction) of the drive shaft. In some embodiments, at least one rigid compression member is located “upstream” from the drive shaft, and at least one rigid compression member is located “downstream” from the drive shaft.

As seen in FIG. 3E, in some embodiments, a major axis of each rigid compression member may be rotated a predetermined angle relative to a major axis of an adjacent rigid compression member. In some embodiments, the predetermined angle may be less than 90 degrees. In some embodiments, the predetermined angle may be 30-60 degrees.

The rigid compression members 370 may be any appropriate shape for intermittently compressing the tube. In some embodiments, the rigid compression members 370 may have an oblong or geometric cross-sectional shape (e.g., when viewed from the central axis 361 of shaft 360). See, e.g., FIG. 3E, where the members are oval in shape. In some embodiments, the rigid compression members 370 have an irregular cross-sectional shape. See, e.g., FIG. 3F, where the rigid compression members have a crescent moon-like shape.

The shaft may extend through a geometric center 362 of each rigid compression member (shown in FIG. 3E as being coaxial with the central axis 361 of shaft 360).

In some embodiments, such as those with irregular shaped rigid compression members, the shaft may extend through a centroid 363 of each rigid compression member.

The shaft may extend through a point offset from the geometric center of each rigid compression member. The shaft may extend through a point offset from the centroid of each rigid compression member. The shaft may extend through a point offset from the centroid and the geometric center of each rigid compression member.

The shaft may be cylindrical.

The shaft may be segmented, and may comprise a plurality of segments. In some embodiments, the segments are coaxial. Referring to FIG. 3G, in some embodiments, a first segment 380 may be offset relative to an adjacent segment 381, such that the adjacent segment is not coaxial with the first segment.

In various aspects, a system may be provided. Referring to FIGS. 1-2B, the system may include circuitry 50. The system may include one or more processors 51. The system may include at least one sensor 52. The system may include an electric motor 55 operably coupled to the one or more processors. The system may include a housing 21 disposed around the one or more processors 51 and the electric motor 55. A drive shaft 56 of the electric motor 55 may extend through an outer surface 27 of the housing (e.g., through an opening (not shown) in the housing). The system may include a purge apparatus 100 as disclosed herein. The purge apparatus may be coupled to the housing such that the drive shaft 56 extends through the opening 113 of the purge apparatus. The pumping mechanism 120 of the purge apparatus may be operably coupled to the drive shaft.

The system may include a purge fluid source 20 (such as a purge bag) containing a purge fluid. The purge bag may be operably coupled to a tube 150. A portion of the tube may be disposed in a tubing channel 350 of the purge apparatus. The system may include a blood pump 10 operably coupled to the purge bag via the tube 150.

The one or more processors 51 may be configured to, collectively, determine a pressure of a fluid in the tube based on a current of the electric motor. The pressure of a fluid in a tube can be determined by measuring the current consumed by the electric motor that is configured to compress the tube. As the motor operates, it draws more current in response to the increasing load caused by fluid pressure within the tube. For instance, if the current to the motor is above a predetermined threshold, this may be indicative of a kink or a clog in the tube. In this instance, the display may instruct a user to check or change the tube. This relationship can be quantified through calibration, where known pressures are correlated with corresponding motor currents. By analyzing the real-time current data with this established relationship, the one or more processors may calculate the pressure in the tube, as higher current indicates greater resistance against compression, reflecting higher pressure.

In some embodiments, determining the pressure of the fluid in the tube may be based on a change in the current of the electric motor. In some embodiments, determining the pressure of the fluid in the tube may be based on the current of the electric motor and information from at least one other sensor. For example, in some embodiments, a database containing a correlation of motor current to pressure may be provided, and configured such that the one or more processors can access the correlation. The one or more processors may be configured to compare the real-time motor current data to the database. The one or more processors may further be able to determine the pressure of the fluid based on a comparison of the motor current to the database containing a correlation of motor current to pressure. The one or more processors may be configured to, collectively, generate an alert or alarm based on the pressure of the fluid in the tube. The one or more processors may be configured to, collectively, generate an alert or alarm if the pressure of the fluid in the tube is outside a predetermined range. The one or more processors may be configured to, collectively, generate an alert or alarm when the current of the electric motor changes from a first value to a second value, the second value being lower than the first value. The one or more processors may be configured to, collectively, generate an alert or alarm when the current of the electric motor changes from a first value to a second value, where the change is sufficiently large (e.g., an absolute value of the difference is greater than a predetermine threshold), or where a directional change is sufficiently large (e.g., if the second value is lower than the first value by at least a predetermined amount).

Referring back to FIG. 2A, the system may include a display 22. The one or more processors 51 may be configured to, collectively, cause the display 22 to show an alert based on a determined motor current or tubing pressure. The one or more processors 51 may be configured to, collectively, cause the display 22 to show a fluid pressure or a motor current. In some embodiments, the one or more processors may be configured to, collectively, cause the display to show a suggested fluid flow rate based on a determined current. In some embodiments, the one or more processors may cause the display to show a warning. For example, the display may show a warning that the fluid source should be empty.

The one or more processors may be configured to, collectively, determine if a liquid is present in the tube based on a current of the electric motor. For example, if the current to the motor is lower than a threshold value, then that may be indicative of no fluid in the tubing. In other examples, in some embodiments, values above a predetermined threshold indicate there is a fluid in the tube. As previously described, the current to the motor is correlated to the amount of fluid in the tubing because of the increased load caused by the fluid pressure in the tube.

The one or more processors may be configured to, collectively, determine a current of the electric motor once every T seconds, where T≥1. In some embodiments, determining the current may include determining an average current of the electric motor over T seconds. In some embodiments, determining the current may include determining a maximum current of the electric motor over T seconds. In some embodiments, determining the current may include determining a minimum current of the electric motor over T seconds. In some embodiments, a change in the current may be measured over T seconds. In some embodiments, the change in the current is used to adjust a flow rate or fluid pressure in the tubing.

The one or more processors may be configured to, collectively, adjust a current of the electric motor based on a determined current of the electric motor. In some embodiments, adjusting a current of the electric motor may include increasing the current if the determined current is below a lower threshold. Adjusting the current may include decreasing the current if the determined current is above an upper threshold. In some embodiments, adjusting the current may include comparing a change in the current over T seconds.

The one or more processors may be configured to, collectively, determine an amount of a liquid fluid passing through the pumping mechanism based on a number of rotations of a wheel of the pumping mechanism. The one or more processors may be configured to, collectively, monitor a total amount of liquid fluid passing through the pumping mechanism over a period of time based on the number of rotations of the wheel of the pumping mechanism, and may be configured to generate an alert or alarm when the total amount of liquid fluid meets or exceeds a predetermined threshold. The predetermined threshold may be based on a volume of a source of the liquid fluid. The one or more processors may be configured to, collectively, display the total amount of fluid on a display operably coupled to the one or more processors. The one or more processors may be configured to, collectively, stop the pumping mechanism when the current of the electric motor changes from a first value to a second value, the second value being lower than the first value.

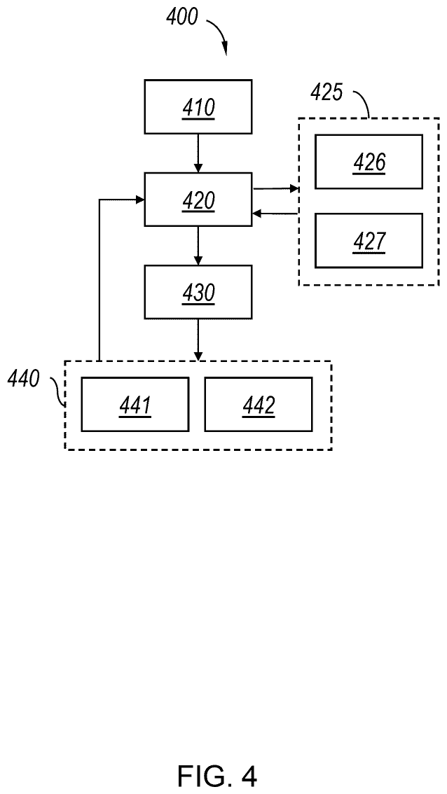

In various aspects, a method may be provided. Referring to FIG. 4, the method 400 may include rotating 410 a drive shaft coupled to an electric motor, causing a pumping mechanism to move liquid fluid from a fluid source through a tube.

The method may include measuring 420 a current to the electric motor while the drive shaft is rotating.

The method may include responding 425 based on the measured current. This may include adjusting 426 a current to the electric motor if the measured current is outside a predetermined range. This may include generating 427 an alert or alarm if the measured current is outside a predetermined range.

The method may include determining 430 a pressure in the tube based on the current to the electric motor. The method may include responding 440 based on the determined pressure. This may include generating 441 an alert or alarm if the determined pressure in the tube is outside a predetermined range. This may include adjusting 442 a current to the electric motor if the measured current is outside a predetermined range.

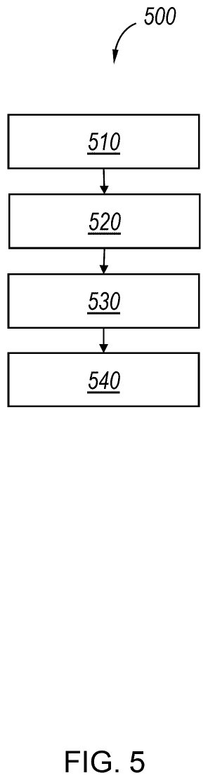

In various aspects, a method may be provided. Referring to FIG. 5, the method 500 may include rotating 510 a drive shaft coupled to an electric motor, causing a pumping mechanism to move liquid fluid from a fluid source through a tube.

The method may include determining 520 a number of rotations of a wheel of the pumping mechanism during a period of time.

The method may include determining 530 a volume of liquid fluid passing through the tube based on the number of rotations.

The method may include generating 540 an alert or alarm if the volume of liquid fluid meets or exceeds a predetermined threshold.

As described, the systems, methods, and apparatus may include one or more processors. The one or more processors may include any microprocessor-type device configured to execute software algorithms and/or instructions. In one embodiment, the one or more processors may include a desktop computer, mainframe, computer system, workstation, image computer, parallel processor, or other computer system (e.g., networked computer) configured to execute a program configured to operate the system. In general, the term “processor” may be broadly defined to encompass any device having one or more processing elements, which execute program instructions (e.g., module) from a non-transitory memory.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

1. A purge apparatus, comprising:
a housing having a hinged lid defining a first surface of the housing, the hinged lid configured to allow access to an internal volume of space of the housing, the housing having an opening on a second surface opposite the first surface, the housing defining a tubing channel configured to receive at least a portion of a tube; and
a pumping mechanism configured to be operably coupled to a motor through the opening, the pumping mechanism being disposed within the internal volume of space of the housing, the pumping mechanism configured to, during operation, compress the tube extending through the purge apparatus so as to continuously maintain a positive pressure downstream of the pumping mechanism.

2. The purge apparatus of claim 1, wherein the tubing channel is configured to, during operation, allow the tube to be pressed against a surface of the hinged lid.

3. The purge apparatus of claim 1, wherein the pumping mechanism comprises a plurality of rollers or wheels, the plurality of rollers or wheels operably coupled together, each roller or wheel spaced an equal distance apart, the plurality of rollers or wheels configured to move within the housing such that, during operation, at least one roller or wheel compresses the tube against an internal surface of the housing at any point in time.

4. The purge apparatus of claim 3, wherein the plurality of rollers or wheels are coupled together via a belt or chain.

5.-9 (canceled)

10. The purge apparatus of claim 1, wherein the pumping mechanism comprises a pump wheel having a plurality of extruding portions, each extruding portion having an equal pitch, the pump wheel configured to rotate within the housing such that, during operation, at least one extruding portion compresses the tube against an internal surface of the housing at any point in time.

11.-15. (canceled)

16. The purge apparatus of claim 10, wherein the pump wheel is configured to be operably connected to the motor via one or more gears disposed within the housing.

17. The purge apparatus of claim 10, wherein a first gear of the one or more gears is configured to receive a drive shaft extending through the opening.

18. (canceled)

19. The purge apparatus of claim 1, wherein the pumping mechanism comprises a plurality of rigid compression members disposed along a shaft, the plurality of rigid compression members configured to provide a pattern of compression to the tube as the shaft rotates.

20. The purge apparatus of claim 19, wherein the shaft is configured to be coupled to one or more worm threads on a drive shaft to cause the shaft to rotate.

21.-22. (canceled)

23. The purge apparatus of claim 19, wherein a major axis of each rigid compression member is rotated a predetermined angle relative to a major axis of an adjacent rigid compression member.

24.-28. (canceled)

29. The purge apparatus of claim 19, wherein the shaft is segmented.

30. A system, comprising:
one or more processors;
an electric motor operably coupled to the one or more processors;
a housing disposed around the one or more processors and the electric motor, a drive shaft of the electric motor extending through an outer surface of the housing;
a purge apparatus of claim 1 coupled to the housing such that the drive shaft extends through the opening of the purge apparatus.

31. The system of claim 30, further comprising a purge bag containing a purge fluid, the purge bag being operably coupled to a tube, a portion of the tube being disposed in a tubing channel of the purge apparatus.

32. The system of claim 31, further comprising a blood pump operably coupled to the purge bag via the tube.

33. The system of claim 30, wherein the one or more processors are configured to, collectively, determine a pressure of a fluid in the tube based on a current of the electric motor.

34. The system of claim 33, wherein determining the pressure of the fluid in the tube is based on a change in the current of the electric motor.

35. The system of claim 33, wherein determining the pressure of the fluid in the tube is based on the current of the electric motor and information from at least one other sensor.

36.-45. (canceled)

46. The system of claim 30, wherein the one or more processors are configured to, collectively, determine an amount of a liquid fluid passing through the pumping mechanism based on a number of rotations of a wheel of the pumping mechanism.

47.-50. (canceled)

51. A method, comprising:
rotating a drive shaft coupled to an electric motor, causing a pumping mechanism to move liquid fluid from a fluid source through a tube, the pumping mechanism being disposed in a housing, at least a portion of the tube being disposed within the housing, the drive shaft extending through a channel in the housing;
measuring a current to the electric motor while the drive shaft is rotating; and
determining a pressure in the tube based on the current to the electric motor.

52.-53. (canceled)

54. A method, comprising:
rotating a drive shaft coupled to an electric motor, causing a pumping mechanism to move liquid fluid from a fluid source through a tube, the pumping mechanism being disposed in a housing, at least a portion of the tube being disposed within the housing, the drive shaft extending through a channel in the housing;
determining a number of rotations of a wheel of the pumping mechanism during a period of time;
determining a volume of liquid fluid passing through the tube based on the number of rotations; and
generating an alert or alarm if the volume of liquid fluid meets or exceeds a predetermined threshold.
